SEQUENCE LISTING (4 sequences)

SEQ ID NO: 1 - DNA, 27 bp, Artificial sequence
  ATP aptamer
acctgggggagtattgcggaggaaggt

SEQ ID NO: 2 - DNA, 14 bp, Artificial sequence
  ATP aptamer extended sequence
accttcctccgcaa

SEQ ID NO: 3 - DNA, 20 bp, Artificial sequence
  CpG-ODN
tccatgacgttcctgacgtt

SEQ ID NO: 4 - DNA, 34 bp, Artificial sequence
  CpG-ODN extended sequence
tccatgacgttcctgacgttaccttcctccgcaa

(19) United States
(12) Patent Application Publication
(10) Pub. No.: US 2024/0148874 A1
(43) Pub. Date: May 9, 2024

(54) IMMUNOADJUVANT-BASED HYDROGEL COMPOSITION AND USE THEREOF

(71) Applicant: SOOCHOW UNIVERSITY, Suzhou (CN)

(72) Inventors: Zhuang LIU, Suzhou (CN); Lele SUN, Suzhou (CN)

(21) Appl. No.: 18/044,565

(22) PCT Filed: Oct. 29, 2020
(86) PCT No.: PCT/CN2020/124668

§ 371 Date: Mar. 8, 2023

(30) Foreign Application Priority Data
Sep. 16, 2020 (CN) 202010976044.3

(51) Int. Cl. A61K 47/36; A61K 47/61; C08L 5/04; C12N 15/115

(52) U.S. Cl. CPC A61K 47/36; A61K 47/61; C08L 5/04; C12N 15/115; C12N 2310/16

(57) ABSTRACT

The invention provides the use of an immunoadjuvant-based hydrogel in the preparing a sensitizing agent for surgical radiotherapy. The present invention also provides an immunoadjuvant-based hydrogel composition comprising sodium alginate, a tumor cell death marker aptamer, and a water-soluble immunoadjuvant with an extended sequence, wherein sodium alginate is covalently linked to the tumor cell death marker aptamer, and the sequence extended from the immunoadjuvant is partially complementary to the sequence of the tumor cell death marker aptamer. The immunoadjuvant-based hydrogel composition can undergo gelation in situ in the presence of calcium ions in the body, and realize the release of immune adjuvants in response to tumor cell death marker during tumor radiotherapy and the long-term intratumoral retention of immune adjuvant in the interval of radiotherapy, thus activating tumor-specific immune response for a long time during radiotherapy, and inhibiting the systemic metastasis of tumors.

This application is the National Stage Application of PCT/CN2020/124668, filed on Oct. 29, 2020, which claims priority to Chinese Patent Application No. 202010976044.3, filed on Sep. 16, 2020, which is incorporated by reference for all purposes as if fully set forth herein.

### FIELD OF THE INVENTION

The present invention relates to the field of therapeutic preparations for tumors, and particularly to an immunoadjuvant-based hydrogel composition and use thereof.

### DESCRIPTION OF THE RELATED ART

Tumor is a malignant disease that threatens human survival. With the aging of the world's population and changes in the living environment and daily habits, the number of deaths caused by cancers rises dramatically every year. In addition to surgical resection, chemotherapy and radiotherapy are currently the main clinical treatments of tumors. In recent years, many studies have found that some chemotherapeutic drugs (anthracyclines and oxaliplatin) and ionizing radiation can induce the immunogenic death of tumor cells that is a unique cell death pathway associated with apoptosis, and the dead cells will release endogenous danger signals. During the process of immunogenic death, autophagy of tumor cells occurs, causing calreticulin (an endoplasmic reticulum calcium-binding protein) to be exposed on the cell surface, and thus stimulating the dendritic cells to engulf tumor antigens. Moreover, high mobility group box B1 (HMGB1) is released from cells, to allow for the formation of stable connections between dendritic cells and dying tumor cells. In the process of immunogenic death, high concentrations of adenosine triphosphate (ATP) produced in metabolism of tumor cells are also largely released, which recruits dendritic cells to tumor foci. These events may trigger an anti-tumor immune response. Due to the above mechanism, in some clinical cases, radiation therapy may sometimes show distant effects, that is, the spontaneous regression of distant metastatic tumors after local tumor radiotherapy.

Currently, there are many problems associated with the chemotherapic agents in clinical development or use, including strong hydrophobicity, low bioavailability, lability, high toxic and side effects, and lack of specificity. Therefore, they cannot fully meet the clinical needs of tumor treatment, etc. Based on the above problems, the use of in-situ hydrogel drug delivery system in tumor therapy has attracted more and more interests of researchers in recent years. The in-situ hydrogel preparations are generally in the form of solutions, suspensions or semi-solids, and the hydrogel system undergoes a phase change immediately after being injected into the administration site, to change from a solution or suspension into a semi-solid or solid. The advantages of this system include local and site-directed effects, extended delivery of drugs, reduced doses of drugs, improved bioavailability, reduced side effects, and improved comfort and compliance of patients. Currently, hydrogel systems are developed by researchers, which gelates in response to temperatures, ions, acoustic waves or light.

Sodium alginate-based hydrogel system is a desirable in-situ drug delivery system for tumors, and a hydrogel system capable of ion-mediated gelation. Sodium alginate is a sodium salt of alginic acid. Alginic acid is a copolymer consisting of a-L-mannuronic acid (M unit) and b-D-guluronic acid (G unit) linked by 1,4-glycosidic bond and comprising different contents of GM, MM and GG segments. The stability, solubility, stickiness and safety of sodium alginate make it a good excipient for pharmaceutical preparations. In an aqueous phase, the carboxyl group of sodium alginate can interact with

coordinates with divalent metal ions such as calcium ions and copper ions, to form a gel. An important advantage of the sodium alginate hydrogel system as an in-situ drug delivery system for tumors is that it can gelate with divalent metal ions such as calcium ions in local tissue fluid of the tumor, so the operation is convenient compared with the pH, temperature- or light-mediated gelation. At present, the use of sodium alginate hydrogel to directly encapsulate a chemotherapeutic agent and an immunoadjuvant for local administration to tumors shows good efficacy in animal tumor models. However, the use of hydrogel encapsulating the immunoadjuvant for enhancing local radiotherapy and radioimmunotherapy has not been reported so far. In particular, the hydrogel system with radiotherapy-responsive immunoadjuvant release function still needs to be developed.

The immunoadjuvant can enhance the immune response by enhancing the antigen processing and presentation efficiency of antigen presenting cells, to significantly improve the immunogenicity of the antigen. It has been demonstrated that the introduction of an immunoadjuvant into tumors in a therapy capable of inducing immunogenic death of tumor cells can effectively enhance the anti-tumor immune response and produce a synergistic therapeutic effect. Since the systemic administration of an immunoadjuvant may cause serious side effects, such as cytokine storm, the immunoadjuvant is often administered directly to the tumor by local injection (e.g. by percutaneous puncture). However, in most clinical cancer treatments, a low-dose chemotherapeutic agent or radiation is repeatedly used, to reduce the side effect. However, during one course of chemotherapy or radiotherapy, multiple percutaneous administration of an immunoadjuvant to a patient with cancer may cause physical and psychological stress to the patient, and it is difficult to give the immunoadjuvant at an optimum time point. Therefore, a smart carrier is needed, with which the long-term retention of the immunoadjuvant in the tumor is realized, and the simultaneous release of the immunoadjuvant is achieved upon administration of the radiotherapy/chemotherapy, so as to achieve a desirable immune stimulation effect.

### SUMMARY OF THE INVENTION

To solve the above technical problems, an objective of the present invention relates to an immunoadjuvant-based hydrogel composition and use thereof. The immunoadjuvant-based hydrogel composition of the present invention gelates in situ in the presence of calcium ions in the body, has tumor cell death marker responsiveness, and simultaneously realizes the release of immune adjuvants in the process of tumor radiotherapy. In the interval of radiotherapy, the long-term intratumoral retention of immune adjuvant is maintained.

A first objective of the present invention is to provide use of an immunoadjuvant-based hydrogel composition in the preparation of a surgical radiotherapy sensitizing agent. The immunoadjuvant-based hydrogel composition comprises sodium alginate and a water-soluble immunoadjuvant encapsulated in sodium alginate.

Preferably, the immunoadjuvant-based hydrogel composition comprises sodium alginate, a tumor cell death marker aptamer, and a water-soluble immunoadjuvant with an extended sequence, where sodium alginate is covalently linked to the tumor cell death marker aptamer by a peptide bond, and the extended sequence is base-complementary to at least a part of the sequence of the tumor cell death marker aptamer.

Preferably, the tumor cell death marker aptamer comprises an ATP aptamer. The nucleotide sequence of the ATP aptamer comprises a sequence as shown in SEQ ID No: 1. The radiotherapy includes multiple low-dose treatments. The radiotherapy sensitizing agent prepared with the immunoadjuvant-based hydrogel composition is responsive to the radiotherapy applied to a tumor, and can achieve the simultaneous release of the immunoadjuvant in the tumor radiotherapy. In the interval of radiotherapy, the long-term intratumoral retention of immune adjuvant is maintained.

Preferably, the nucleotide sequence of the extended sequence comprises a sequence as shown in SEQ ID No: 2.

Preferably, the water-soluble immunoadjuvant is selected from the group consisting of a polynucleotide, CpG oligodeoxynucleotide, polyinosinic acid, polyICLC, lipopolysaccharide, muramyl peptide, lipoid A, a cytokine and any combination thereof. More preferably, the water-soluble immunoadjuvant includes CpG oligodeoxynucleotide.

Preferably, the surgical radiotherapy sensitizing agent is used for the treatment of solid tumors. Preferably, the solid tumors include one or more of colon cancer, melanoma, breast cancer, lung cancer, and head and neck cancer. Preferably, the radiotherapy sensitizing agent is used under irradiation at a single dose of 0.5-10 Gray, with a total radiation dose of 5-80 Gray.

Preferably, the radiotherapy sensitizing agent is administered by intravenous injection.

Preferably, the viscosity of sodium alginate is 5-1000 Cp. More preferably, the viscosity of sodium alginate is 50-200 Cp.

A second objective of the present invention is to provide an immunoadjuvant-based hydrogel composition, which comprises sodium alginate, a tumor cell death marker aptamer, and a water-soluble immunoadjuvant with an extended sequence, where sodium alginate is covalently linked to the tumor cell death marker aptamer by a peptide bond, and the extended sequence is base-complementary to at least a part of the sequence of the tumor cell death marker aptamer. Preferably, the tumor cell death marker aptamer comprises an ATP aptamer. The nucleotide sequence of the ATP aptamer comprises a sequence as shown in SEQ ID No: 1.

Preferably, the nucleotide sequence of the extended sequence comprises a sequence as shown in SEQ ID No: 2.

Preferably, the water-soluble immunoadjuvant is selected from the group consisting of a polynucleotide, CpG oligodeoxynucleotides, polyinosinic acid, polyICLC, lipopolysaccharide, muramyl peptide, lipoid A, a cytokine and any combination thereof.

Preferably, the water-soluble immunoadjuvant includes CpG oligodeoxynucleotides (CpG-ODN). CpG-ODN is an oligodeoxyribonucleic acid (DNA) sequence that can enhance the function of antigen-presenting cells.

Preferably, the viscosity of sodium alginate is 5-1000 Cp. Sodium alginate has a structure shown below:

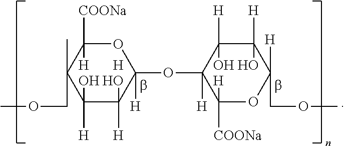

The immunoadjuvant-based hydrogel composition of the present invention comprises sodium alginate, where the carboxyl group of sodium alginate can form a hydrogel mediated by calcium ions in situ in the tumor, and the immunoadjuvant is encapsulated in the gel. The use of the hydrogel allows for the local slow release of the immunoadjuvant in the tumor, to reduce the doses of the drug. However, only the presence of a high concentration of immunoadjuvant at the time of tumor cell death (tumor antigen release) can elicit an optimal anti-tumor immune response. Although a conventional hydrogel system allows the immunoadjuvant to release slowly, such release is not controlled, and it is difficult to ensure that a suitable concentration of immunoadjuvant is present locally in the tumor at the optimal time point. Since the tumor cell death marker aptamer in the composition of the present invention is linked to the water-soluble immunoadjuvant through base pairing, the radiation therapy, when applied to tumors, causes the death of tumor cells to release a tumor cell death marker (such as ATP). The tumor cell death marker binds more potently to the tumor cell death marker aptamer, such that the water-soluble immunoadjuvant is released from the hydrogel, and thus a higher concentration of immunoadjuvant is locally present in the tumor during the production of the tumor antigen to produce an endogenous tumor vaccine in situ. In the absence of radiotherapy, the immunoadjuvant is not released and remains in the tumor for a long time. Therefore, the immunoadjuvant-based hydrogel composition is useful in the preparation of a radiotherapy sensitizing agent to additionally enhance the antitumor immune responses elicited by multiple low-dose radiotherapy.

A third objective of the present invention is to provide a method for preparing an immunoadjuvant-based hydrogel composition, which comprises the following steps:
(1) reacting sodium alginate and an amino-modified tumor cell death marker aptamer in a solution, to obtain a sodium alginate-ATP aptamer conjugate, where the tumor cell death marker aptamer is preferably an ATP aptamer, and has a nucleotide sequence comprising a sequence as shown in SEQ ID No: 2;
(2) subjecting the sodium alginate-ATP aptamer conjugate to a DNA hybridization reaction with a water-soluble immunoadjuvant with an extended sequence in a buffer, to obtain the immunoadjuvant-based hydrogel composition, where the extended sequence is base-complementary to at least a part of the sequence of the tumor cell death marker aptamer.

Preferably, in Step (1), the molar ratio of the carboxyl group in sodium alginate to the amino group in the amino-modified tumor cell death marker aptamer is 100-5000: 1. More preferably, the molar ratio of the carboxyl group in sodium alginate to the amino group in the amino-modified tumor cell death marker aptamer is 1000-1100: 1.

Preferably, in Step (1), sodium alginate is activated before reaction with the amino-modified tumor cell death marker aptamer.

Preferably, sodium alginate is activated with EDC under an acidic condition, and then the activated sodium alginate is reacted with the amino-modified tumor cell death marker aptamer under an alkaline condition. Specifically, Step (1) includes the following steps:
(S1) mixing an aqueous solution of sodium alginate with an aqueous solution of the amino-modified tumor cell death marker aptamer, adjusting the pH of the mixed solution to 4-6, then adding EDC to the mixed solution, and reacting at 37° C.; and
(S2) adding a sodium acetate solution to the product obtained in Step (S1), mixing uniformly, then adding ethanol, and reacting at −80° C., to isolate a sodium alginate-ATP aptamer conjugate from the aqueous solution.

Preferably, in Step (S1), the aqueous solution of sodium alginate has a concentration of 0.1-0.2 mg/mL.

Preferably, in Step (S1), the aqueous solution of the amino-modified tumor cell death marker aptamer has a concentration of 0.1 mmol/L.

During the preparation process of the immunoadjuvant-based hydrogel composition of the present invention, the amino-modified tumor cell death marker aptamer is linked to sodium alginate through a covalent bond formed by the amino group on the amino-modified tumor cell death marker aptamer with the carboxyl group on sodium alginate, where the reaction is mediated by an activator. The water-soluble immunoadjuvant is assembled to sodium alginate by DNA hybridization of the extended sequence with the tumor cell death marker aptamer. The water-soluble immunoadjuvant is released from sodium alginate, because the tumor cell death marker specifically binds to the tumor cell death marker aptamer, thus opening the DNA duplex formed by the water-soluble immunoadjuvant and the tumor cell death marker aptamer.

Preferably, in Step (1), the amino is linked to the 5′ end of the tumor cell death marker aptamer. Preferably, in Step (1), the amino-modified tumor cell death marker aptamer includes the tumor cell death marker aptamer and the amino group at one end thereof. The tumor cell death marker aptamer is an oligodeoxyribonucleic acid (DNA) sequence screened by systematic evolution of ligands by exponential enrichment (SELEX) technology. The sequence is specifically 5′-acctgggggagtattgcggaggaaggt-3′ (SEQ ID No:1), and this sequence can specifically bind to ATP to form a certain three-dimensional structure.

In Step (2), the water-soluble immunoadjuvant with an extended sequence is preferably CpG-ODN with an extended sequence. CpG-ODN is an oligodeoxyribonucleic acid (DNA) sequence that can enhance the function of antigen-presenting cells, and has a sequence of 5′-tccatgacgttcctgacgtt-3′ (SEQ ID No:3), where an extended sequence is present at the 3′ end, and the extended sequence is complementary to a part of the ATP aptamer: 5′-accttcctccgcaa-3′ (SEQ ID No:2).

For example, the tumor cell death marker aptamer is ATP aptamer. As shown in FIG. 1, CpG-ODN with an extended sequence could hybridize with sodium alginate-ATP aptamer to form a smart sodium alginate hydrogel preparation capable of slow release of immunoadjuvant in responsive to adenosine triphosphate (ATP). When the preparation is injected into tumors, a hydrogel is formed as mediated by calcium ions in the tumor, to encapsulate the immunoadjuvant therein. When the tumor receives chemotherapy or radiotherapy, a large amount of ATP is released, and a tumor vaccine is formed. ATP released by the tumor binds to the ATP aptamer, to release the immunoadjuvant. The dendritic cells in the tumor become mature after uptake of the tumor vaccine and immunoadjuvant, and subsequently migrate to lymph nodes to stimulate the antigen-specific T cell activation. The activated antigen-specific T cells then kill the tumor.

By virtue of the above solutions, the present invention has at least the following advantages.

The present invention provides a hydrogel composition with immunoadjuvant slow-release function, which can rapidly gelate as mediated by calcium ions in the aqueous phase, and slowly release the water-soluble immunoadjuvant in response to the tumor cell death marker.

The immunoadjuvant-based hydrogel composition of the present invention can be used to prepare a radiotherapy sensitizing agent. Compared with systemic drug delivery, the locally slow release of the immunoadjuvant by the hydrogel in the tumor can extent the retention of the adjuvant in the tumor site, reduce the adjuvant dosage, increase the bioavailability of the adjuvant, and reduce the side effects, thus well enhancing the anti-tumor immune response induced by local radiotherapy. The release of immunoadjuvant is controlled by the signaling molecule released upon tumor cell death, whereby a high concentration of immunoadjuvant is locally present when the tumor antigen is produced. The antigen in combination with the immunoadjuvant can well stimulate antigen presenting cells, to induce a more potent anti-tumor immune response, and achieve synergistic effect.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

FIG. 1 shows the working principle of a smart sodium alginate hydrogel formulation capable of adenosine triphosphate (ATP)-responsive slow release of immunoadjuvant.

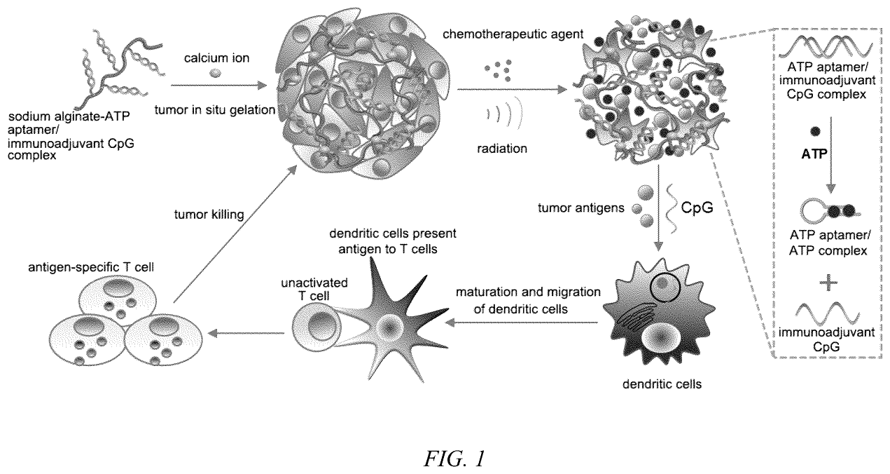

FIG. 2 shows the results of chemotherapy sensitization in a colon cancer subcutaneous tumor mouse model with a sodium alginate hydrogel radiotherapy/chemotherapy sensitizing agent encapsulating the immunoadjuvant CpG-ODN in Example 1.

FIG. 3 shows the result of electrophoresis analysis of sodium alginate-ATP aptamer conjugate in Example 2, in which: Lane 1 is ATP aptamer, and Lanes 2-7 are sodium alginate-ATP aptamer conjugates.

FIG. 4 shows the result of electrophoresis analysis of a smart sodium alginate hydrogel formulation capable of adenosine triphosphate (ATP)-responsive slow release of the immunoadjuvant in Example 2, in which Lane 1 is ATP aptamer (A-Apt), Lane 2 is CpG-ODN, and Lane 3 is a duplex (A-Apt/CpG) formed of ATP aptamer and CpG-ODN, Lanes 4, 5, and 6 are respectively hybridization products of sodium alginate-ATP aptamer conjugate with CpG-ODN at various ratios (ALG-A-Apt/CpG(Apt:CpG=2:1), ALG-A-Apt/CpG(Apt:CpG=1:1), ALG-A-Apt/CpG(Apt:CpG=1:2)), and Lane 7 is sodium alginate-ATP aptamer conjugate (ALG-A-Apt).

FIG. 5 shows of the test results of ATP-responsive release of the immunoadjuvant CpG-ODN in Example 2.

FIG. 6 shows the results of chemotherapy sensitization with a smart sodium alginate hydrogel preparation capable of ATP-responsive slow release of the immunoadjuvant in a colon cancer subcutaneous tumor mouse model in Example 3.

FIG. 7 shows the results of immune response evaluation in chemotherapy sensitization with a smart sodium alginate hydrogel preparation capable of ATP-responsive slow release of the immunoadjuvant in a colon cancer subcutaneous tumor mouse model in Example 4;

FIG. 8 shows the results of evaluating the immune memory function in cured mice in chemotherapy sensitization with a smart sodium alginate hydrogel preparation capable of ATP-responsive slow release of the immunoadjuvant in a colon cancer subcutaneous tumor mouse mode in Example 5.

FIG. 9 shows the results of radiotherapy sensitization with a smart sodium alginate hydrogel preparation capable of ATP-responsive slow release of the immunoadjuvant in a colon cancer subcutaneous tumor mouse model in Example 6.

FIG. 10 shows the results of radiotherapy sensitization with a smart sodium alginate hydrogel preparation capable of ATP-responsive slow release of the immunoadjuvant in a melanoma mouse model in Example 7.

FIG. 11 shows the distal tumor inhibitory effect enhanced with a smart sodium alginate hydrogel preparation capable of ATP-responsive slow release of the immunoadjuvant in combination with a immune checkpoint inhibitor in an external radiotherapy in Example 8.

FIG. 12 shows the results of a smart sodium alginate hydrogel preparation capable of ATP-responsive slow release of the immunoadjuvant in combination with an immune checkpoint inhibitor for enhancing the radiotherapeutic effect for primary breast cancer tumors in Example 9.

### DETAILED DESCRIPTION OF THE PREFERRED EMBODIMENTS

The specific embodiments of the present invention will be described in further detail by way of examples. The following examples are intended to illustrate the present invention, instead of limiting the scope of the present invention.

### Example 1: Preparation and Use of a Sodium Alginate Hydrogel Radiotherapy/Chemotherapy Sensitizing Agent Encapsulating the Immunoadjuvant CpG-ODN

(1) 400 mg of sodium alginate was weighed, and dissolved in 9.6 mL of sterile deionized water, by repeatedly shaking, to prepare a 4% sodium alginate aqueous solution.
(2) CpG oligodeoxynucleotide (CpG-ODN, having a nucleotide sequence as shown in SEQ ID No: 3) as a dry powder was dissolved in sterile deionized water, to give a final concentration is 1 mmol/L.
(3) The solutions in Step (1) and Step (2) were mixed at a certain ratio, and deionized water was added to adjust the final concentration of sodium alginate to 10-20 mg/mL. In this way, a sodium alginate hydrogel radiotherapy/chemotherapy sensitizing agent encapsulating the immunoadjuvant CpG-ODN was obtained.

The sodium alginate hydrogel radiotherapy/chemotherapy sensitizing agent encapsulating the immunoadjuvant CpG-ODN was used for chemotherapy sensitization in a mouse colon cancer subcutaneous tumor model. The treatment was started on day 7 after inoculation of the mouse CT26 colon cancer tumor. Tumor-bearing BALB/c mice were randomly divided into the following 4 groups (6 mice in each group): Group 1: control group (untreated); Group 2: intratumoral injection of sodium alginate and intravenous injection of oxaliplatin (ALG+OxPt);

Group 3: intratumoral injection of sodium alginate hydrogel radiotherapy/chemotherapy sensitizing agent encapsulating the immunoadjuvant CpG-ODN (ALG/CpG); Group 4: intratumoral injection of sodium alginate hydrogel radiotherapy/chemotherapy sensitizing agent encapsulating the immunoadjuvant CpG-ODN, and intravenous injection of oxaliplatin (ALG/CpG+OxPt). The final concentration of sodium alginate in each treatment group was 10 mg/mL, the volume of intratumoral injection was 25 μL, and the dose of CpG-ODN per mouse was 15 μg. On days 7, 10, 13 and 16, oxaliplatin was administered intravenously at a dose of 3 mg/kg body weight. After respective treatments for the mice, the tumor growth was measured.

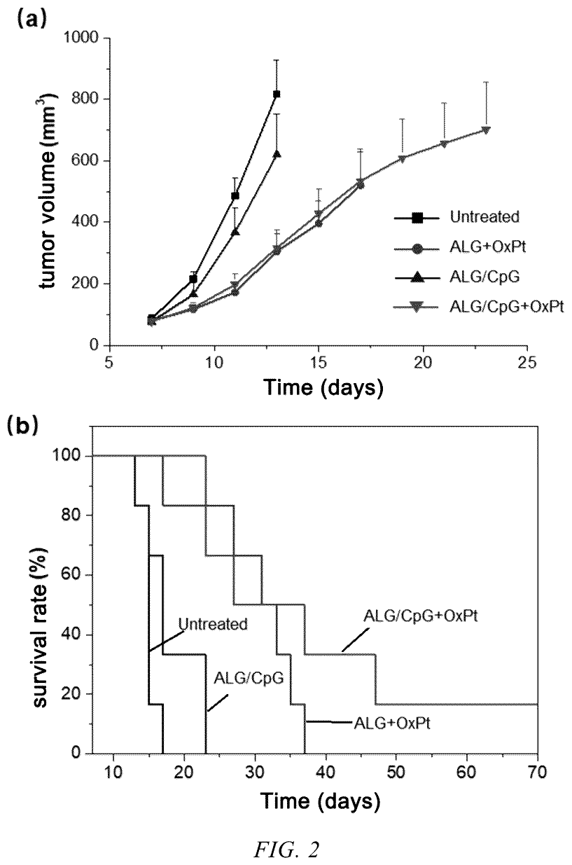

FIGS. 2(a) and (b) shows the tumor volume and mouse survival rate vs time during the experiment, respectively. The results show (FIG. 2) that compared with the control group, the tumor growth in Group 3 was only partially inhibited, the tumor growth in Group 2 was significantly inhibited, and the tumor growth in Group 4 was more significantly inhibited, with ⅙ of the tumor being completely cleared. The results show that the sodium alginate hydrogel radiotherapy/chemotherapy sensitizing agent encapsulating the immunoadjuvant CpG-ODN can enhance the chemotherapeutic effect to some extent.

### Example 2: Preparation of Smart Sodium Alginate Hydrogel Formulation Capable of ATP-Responsive Slow Release of Immunoadjuvant

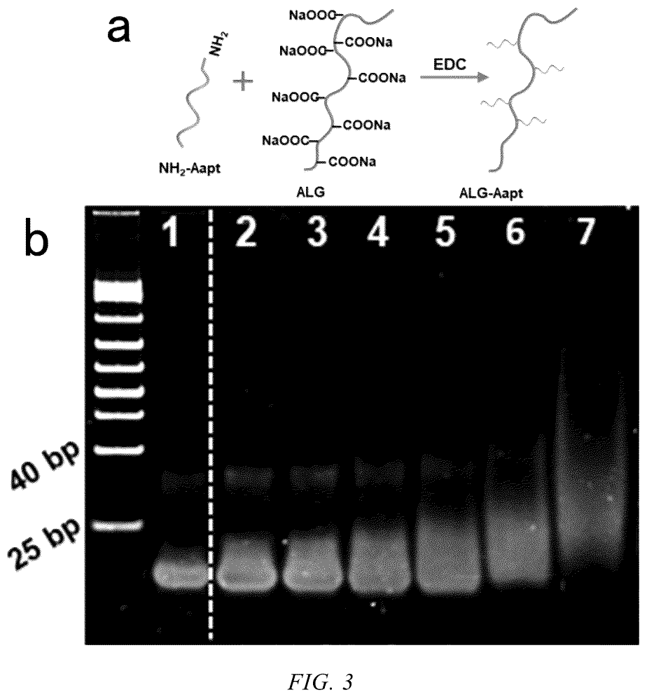

(1) Preparation of sodium alginate-ATP aptamer conjugate: 0.2 mg/mL low-viscosity sodium alginate (with a viscosity of 20-100 Cp) and 0.1 mmol/L ATP aptamer modified with a single amino group at the 5′ end (SEQ ID No: 1:5′-acctgggggagtattgcggaggaaggt-3′) were mixed, where the molar ratio of the carboxyl group in sodium alginate to the amino group in the ATP aptamer was 1000:1. The mixed solution was adjusted to pH 5 with 5× concentrated MES buffer.
(2) EDCHC1 powder of required weight was quickly added to the mixed solution, where the molar ratio of EDCHC1 to the hydroxyl group in sodium alginate was 100:1. Then, the reaction was carried out at 37° C. with continuous shaking.
(3) A 3M sodium acetate solution was added at a volume ratio of ⅛ to the reaction solution obtained in Step (2). Then ethanol was added, in a volume that was 3.75 times the volume of the aforementioned mixture. The solution was allowed to stay at −80° C. for 10 min and then centrifuged at 10,000 g for 10 min at 4° C.
(4) The precipitate obtained by centrifugation was dissolved in deionized water, and Step (3) was repeated 1 time. The resulting precipitate was the sodium alginate-ATP aptamer conjugate, which was dissolved in a phosphate buffer. The reaction principle is shown in FIG. 3a.

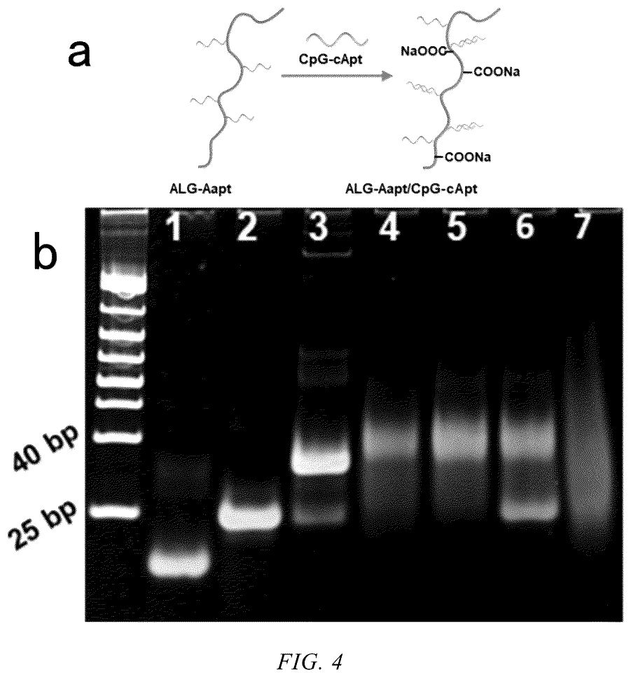

The sodium alginate-ATP aptamer conjugate was analyzed by native polypropylene amide gel electrophoresis. The results (FIG. 3b) show compared with ATP aptamer, the sodium alginate-ATP aptamer conjugate prepared in Example 1 exhibits obvious hysteresis in electrophoretic migration, indicating that sodium alginate is conjugated to the ATP aptamer, and the molecular weight increases, causing the electrophoretic mobility to slow down.
(5) Assembly of CpG-ODN having extended sequence with sodium alginate-ATP aptamer conjugate CpG-ODN with an extended sequence has a sequence as shown in SEQ ID No: 4: 5′-tccatgacgttcctgacgttaccttcctccgcaa -3′. CpG-ODN with an extended sequence was dissolved in a phosphate buffer, to give a concentration of 0.1 mM. The obtained solution was mixed with the sodium alginate-ATP aptamer conjugate, and reacted in a freezer at 4° C. for over 2 hrs, where the molar ratio of CpG-ODN with an extended sequence to the ATP aptamer was 1:2. The obtained sample was the smart sodium alginate hydrogel formulation capable of slow releasing immunoadjuvant in responsive to ATP. The reaction principle is shown in FIG. 4a.

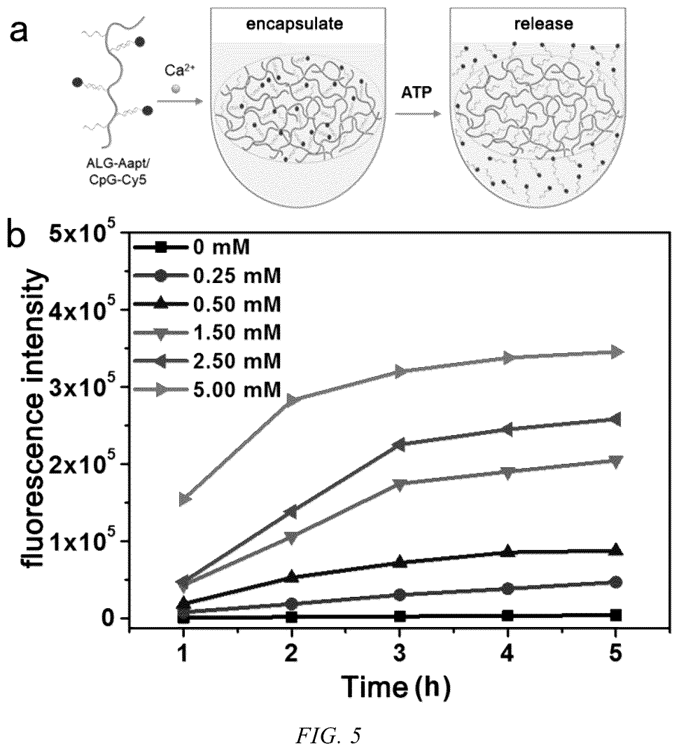

The smart sodium alginate hydrogel formulation was analyzed by native polypropylene amide gel electrophoresis. The results (FIG. 4b) show that compared with the sodium alginate-ATP aptamer conjugate, the smart sodium alginate hydrogel formulation exhibits obvious hysteresis in electrophoretic migration, indicating that CpG-ODN with an extended sequence is assembled to the sodium alginate molecule by hybridization with the ATP aptamer, so the molecular weight of the smart sodium alginate hydrogel preparation becomes larger, causing the electrophoretic mobility to slow down.
(6) Test of ATP-responsive release of immunoadjuvant CpG-ODN: To monitor the release of CpG-ODN from the hydrogel, CpG-ODN modified with a fluorophore at one end was used. According to Examples 1 and 3, a smart sodium alginate hydrogel formulation capable of ATP-responsive slow release of immunoadjuvant was prepared. The prepared sodium alginate hydrogel preparation was mixed with a 4 wt % sodium alginate solution, to give a final concentration of sodium alginate of 1%. The mixture was slowly transferred into a calcium chloride solution with a concentration of 10 mmol/L. 5 minutes later, the calcium chloride solution was removed. A calcium chloride solution containing a certain concentration of ATP was added, and the solution was taken out every 1 hr for fluorescence quantification. Then, the same volume of calcium chloride solution containing a certain concentration of ATP was supplemented. The release principle is shown in FIG. 5a.

The test results (FIG. 5b) show that ATP can trigger the release of CpG-ODN from the sodium alginate hydrogel, and the higher the ATP concentration is, the faster the release rate will be.

### Example 3: Chemotherapy Sensitization with Smart Sodium Alginate Hydrogel Preparation Capable of ATP-Responsive Slow Release of Immunoadjuvant in Colon Cancer Subcutaneous Tumor Mouse Model

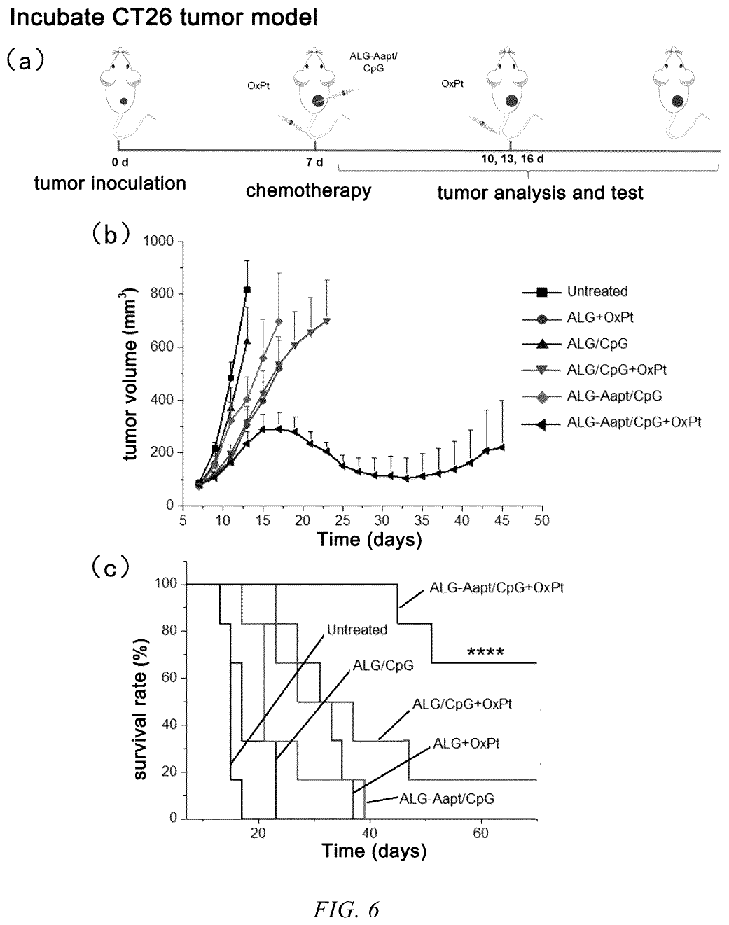

The mice were treated on day 7 after the inoculation of CT26 colon cancer tumor. Tumor-bearing BALB/c mice were randomly divided into the following 6 groups (6 mice in each group): Group 1: control group (untreated); Group 2: intratumoral injection of sodium alginate and intravenous injection of oxaliplatin (ALG+OxPt); Group 3: intratumoral injection of mixture of sodium alginate with CpG-ODN (ALG/CpG); Group 4: intratumoral injection of mixture of sodium alginate and CpG-ODN and intravenous injection of oxaliplatin (ALG/CpG+OxPt); Group 5: intratumoral injection of smart sodium alginate hydrogel preparation (ALG-Aapt/CpG) capable of ATP-responsive slow release of immunoadjuvant obtained in Step (5) of Example 2; Group 6: intratumoral injection of smart sodium alginate hydrogel preparation capable of ATP-responsive slow release of immunoadjuvant obtained in Step (5) of Example 2, and intravenous injection of oxaliplatin (ALG-Aapt/CpG+OxPt). The final concentration of sodium alginate in each treatment group was 10 mg/mL, the volume of intratumoral injection was 25 μL, and the dose of CpG-ODN per mouse was 15 μg. On days 7, 10, 13 and 16, oxaliplatin was administered intravenously at a dose of 3 mg/kg body weight. After respective treatments for the mice, the tumor growth was measured. The treatment process is schematically shown in FIG. 6(a).

FIGS. 6 (b) and (c) show the tumor volume and mouse survival rate vs time during the experiment, respectively. The results show (FIG. 6) that compared with the control group, the tumor growth in Group 2, Group 3, Group 4 and Group 5 was only partially inhibited, and the tumor growth in Group 6 was more significantly inhibited, where the tumor of some mice was completely regressed. This suggests that the smart sodium alginate hydrogel formulation capable of ATP-responsive slow release of immunoadjuvant in combination of a low dose of chemotherapy can efficiently and synergistically kill the tumor.

### Example 4: Immune Response Evaluation in Chemotherapy Sensitization with Smart Sodium Alginate Hydrogel Preparation Capable of ATP-Responsive Slow Release of Immunoadjuvant in Colon Cancer Subcutaneous Tumor Mouse Model

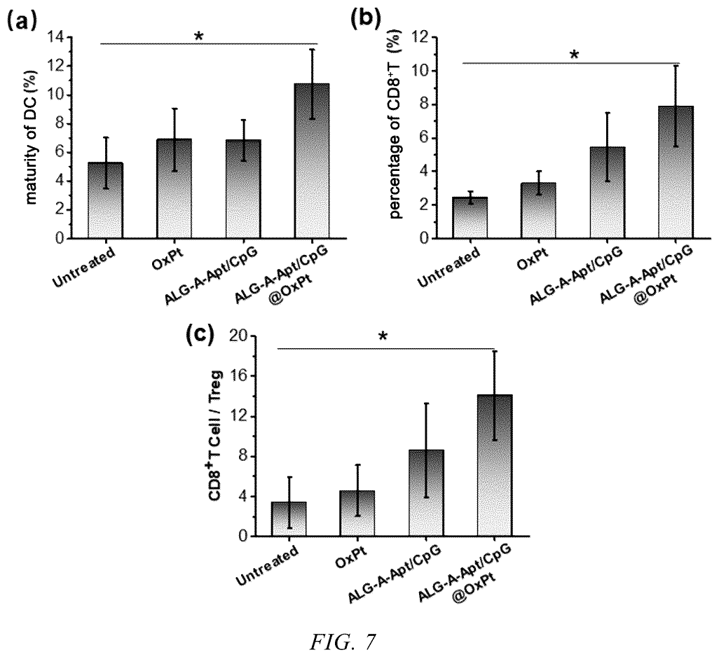

The mice were treated on day 7 after the inoculation of CT26 colon cancer tumor. Tumor-bearing BALB/c mice were randomly divided into the following 4 groups (6 mice in each group): Group 1: control group (untreated); Group 2: intratumoral injection of sodium alginate and intravenous injection of oxaliplatin (OxPt); Group 3: intratumoral injection of smart sodium alginate hydrogel preparation (ALG-A-Apt/CpG) capable of ATP-responsive slow release of immunoadjuvant obtained in Step (5) of Example 2; Group 4: intratumoral injection of smart sodium alginate hydrogel preparation capable of ATP-responsive slow release of immunoadjuvant obtained in Step (5) of Example 2 and intravenous injection of oxaliplatin (ALG-A-Apt/CpG@OxPt). The final concentration of sodium alginate in each treatment group was 10 mg/mL, the volume of intratumoral injection was 25 μL, and the dose of CpG-ODN per mouse was 15 μg. On days 7, 10, 13 and 16, oxaliplatin was administered intravenously at a dose of 3 mg/kg body weight. On day 5 after the treatment, mice in each group were sacrificed, and the maturity of dendritic cells in inguinal lymph nodes, percentage of CD8 positive T cells in the tumor, and ratio of CD8 positive T cells to regulatory T cells was identified. The evaluation results show (FIG. 7) that compared with the control group, the maturity of dendritic cells (DC) in lymph nodes, percentage of CD8 positive T cells in the tumor, and ratio of CD8 positive T cells to regulatory T cells are not significantly improved in Group 2 and Group 3; and the maturity of dendritic cells (DC) in lymph nodes, percentage of CD8 positive T cells in the tumor, and ratio of CD8 positive T cells to regulatory T cells are significantly improved in Group 4 after treatment. This suggests that the smart sodium alginate hydrogel formulation capable of ATP-responsive slow release of immunoadjuvant can efficiently enhance the anti-tumor effect induced by multiple low doses of chemotherapy.

### Example 5: Evaluation of Immune Memory Function in Cured Mice in Chemotherapy Sensitization with Smart Sodium Alginate Hydrogel Preparation Capable of ATP-Responsive Slow Release of Immunoadjuvant in Colon Cancer Subcutaneous Tumor Mouse Model

70 days after the treatment, peripheral blood was collected from mice with complete tumor regression (Cured group) in Example 4 and memory CD8-positive T cells were identified. The mouse CT26 colon tumor was then subcutaneously re-inoculated into mice with complete tumor regression, and untreated mice (Naive group) in a control group. The tumor growth was monitored after tumor inoculation.

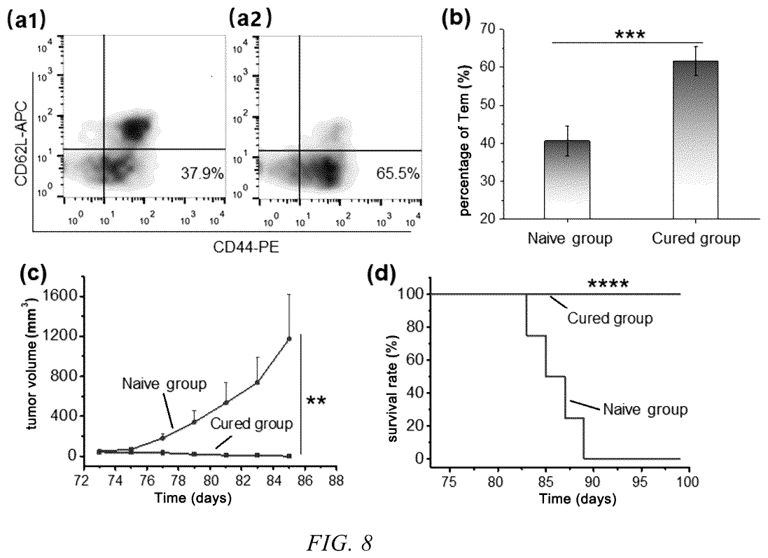

FIGS. 8(a1), (a2), and (b) shows the immune evaluation results. FIGS. 8(c) and (d) shows the tumor volume and mouse survival rate vs time during the experiment, respectively. The evaluation shows that the percentage of CD8-positive effector memory T cells in peripheral blood of mice with complete tumor regression (FIG. 8 (a2) and (b)) is significantly higher than that of untreated mice of the same age (FIG. 8 (a1) and (b)). No obvious tumor growth is found in mice with complete tumor regression, but the tumor grows rapidly on the body of untreated mice. This shows that effective immune memory is developed in mice cured by chemotherapy combined with the smart sodium alginate hydrogel formulation capable of ATP-responsive slow release of immunoadjuvant.

### Example 6: Radiotherapy Sensitization with Smart Sodium Alginate Hydrogel Preparation Capable of ATP-Responsive Slow Release of Immunoadjuvant in Colon Cancer Subcutaneous Tumor Mouse Model

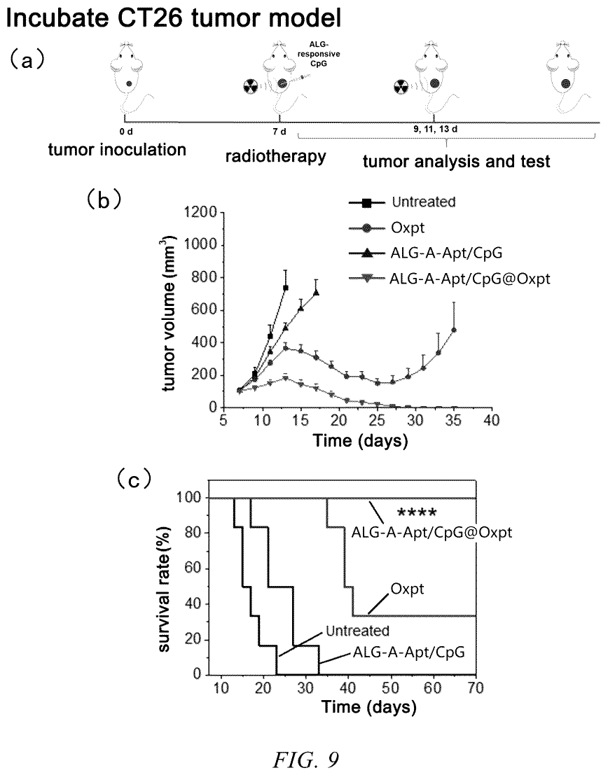

The mice were treated on day 7 after the inoculation of CT26 colon cancer tumor. Tumor-bearing BALB/c mice were randomly divided into the following 4 groups (6 mice in each group): Group 1: control group (Unteated); Group 2: intratumoral injection of sodium alginate and local X-ray irradiation of tumor (at a dose of 8 Gray) (RT); Group 3: intratumoral injection of smart sodium alginate hydrogel preparation capable of ATP-responsive slow release of immunoadjuvant obtained in Step (5) in Example 2 (ALG-Aapt/CpG); Group 4: intratumoral injection of smart sodium alginate hydrogel preparation capable of ATP-responsive slow release of immunoadjuvant obtained in Step (5) of Example 2 and local X-ray irradiation of tumor (at a dose of 8 Gray)(ALG-Aapt/CpG+RT). The final concentration of sodium alginate in each treatment group was 10 mg/mL, the volume of intratumoral injection was 25 μL, and the dose of CpG-ODN per mouse was 15 μg. On days 7, 9, 11 and 13, the mice were irradiated at a dose of 2 Gray per tumor. The treatment process is schematically shown in FIG. 9(a).

FIGS. 9(b) and (c) shows the tumor volume and mouse survival rate vs time during the experiment, respectively. The experimental results show (FIG. 9) that compared with the control group, the tumor growth in Group 2 and Group 3 was only partially inhibited, and the tumor growth in Group 4 was more significantly inhibited, where the tumor of all mice was completely regressed. This suggests that the smart sodium alginate hydrogel formulation capable of ATP-responsive slow release of immunoadjuvant in combination of a low dose of radiotherapy can efficiently and synergistically kill the tumor.

### Example 7: Radiotherapy Sensitization with Smart Sodium Alginate Hydrogel Preparation Capable of ATP-Responsive Slow Release of Immunoadjuvant in Melanoma Mouse Model

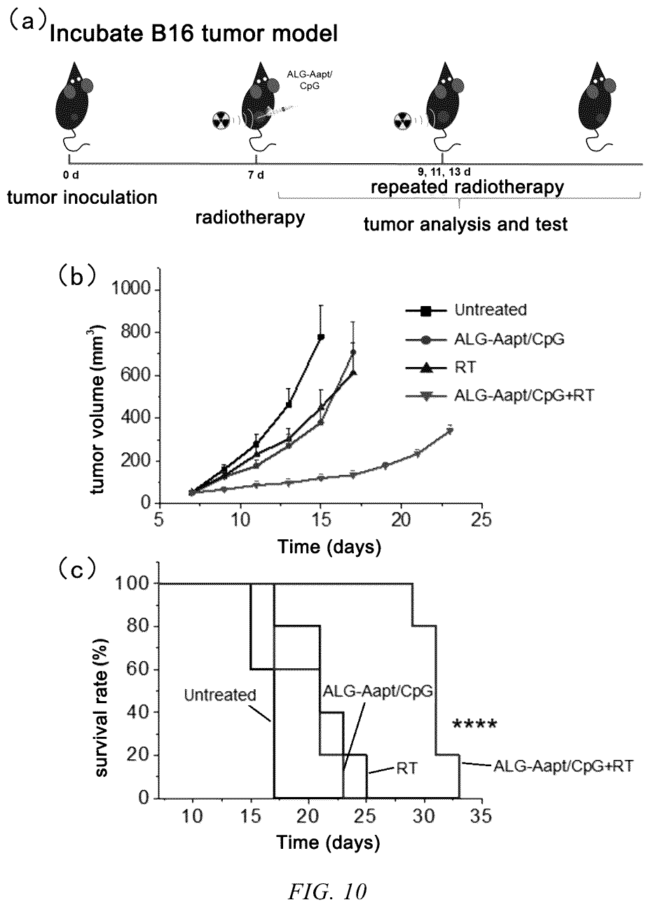

The mice were treated on day 7 after the inoculation of B16 melanoma. Tumor-bearing C57 mice were randomly divided into the following 4 groups (6 mice in each group): Group 1: control group (untreated); Group 2: intratumoral injection of sodium alginate and local irradiation of tumor with X-ray at a dose of 8 Gray (RT); Group 3: intratumoral injection of smart sodium alginate hydrogel preparation capable of ATP-responsive slow release of immunoadjuvant obtained in Step (5) in Example 2 (ALG-Aapt/CpG); Group 4: intratumoral injection of smart sodium alginate hydrogel preparation capable of ATP-responsive slow release of immunoadjuvant obtained in Step (5) of Example 2 and local irradiation of tumor with X-ray at a dose of 8 Gray (ALG-Aapt/CpG+RT). The final concentration of sodium alginate in each treatment group was 10 mg/mL, the volume of intratumoral injection was 25 μL, and the dose of CpG-ODN per mouse was 15 μg. On days 7, 9, 11 and 13, the mice were irradiated at a dose of 2 Gray per tumor. The treatment process is schematically shown in FIG. 10(a).

FIGS. 10(b) and (c) respectively show the tumor volume and mouse survival rate vs time during the experiment. The experimental results show (FIG. 10) that compared with the control group, the tumor growth in Group 2 and Group 3 was only partially inhibited, the tumor growth in Group 4 was more significantly inhibited, and the survival period of mice was effectively prolonged. This suggests that the smart sodium alginate hydrogel formulation capable of ATP-responsive slow release of immunoadjuvant in combination of a low dose of radiotherapy can efficiently inhibit melanoma.

### Example 8: Use of Smart Sodium Alginate Hydrogel Formulation Capable of ATP-Responsive Slow Release of Immunoadjuvant in Enhancing Distal Tumor Inhibitory Effect of External Radiotherapy

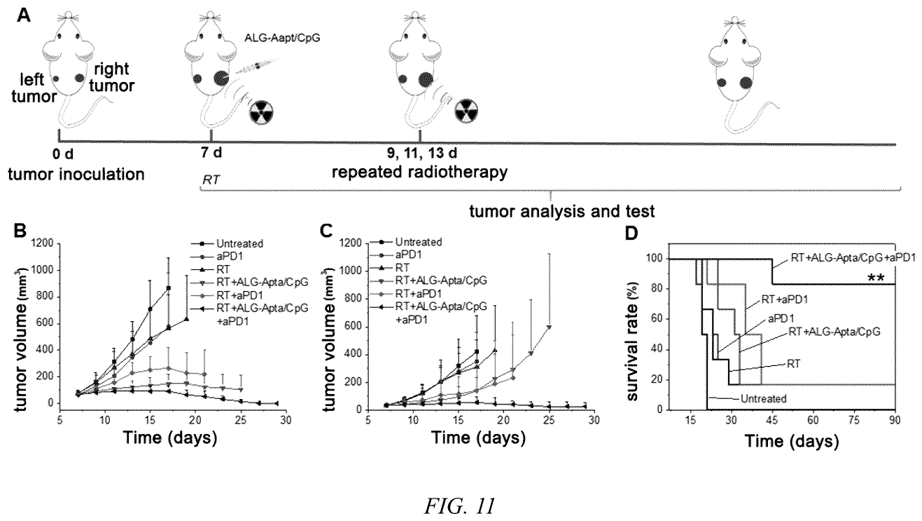

As shown, two colon cancer tumors were inoculated subcutaneously on both sides of the back of the mice, and treatment was started on Day 7 after inoculation. Tumor-bearing BALB/c mice were randomly divided into the following 6 groups (6 mice in each group): Group 1: control group (Unteated); Group 2: intravenous injection of immune checkpoint inhibitor PD1 antibody (aPD1); Group 3: local X-ray irradiation of tumor (at a dose of 8 Gray) (RT); Group 4: intratumoral injection of smart sodium alginate hydrogel preparation (ALG-Aapt/CpG) capable of ATP-responsive slow release of immunoadjuvant obtained in Step (5) in Example 2 and local X-ray irradiation of tumor (at a dose of 8 Gray) (RT); Group 5: intravenous injection of immune checkpoint inhibitor PD1 antibody (aPD1) and local X-ray irradiation of tumor (at a dose of 8 Gray) (RT); Group 6: intratumoral injection of smart sodium alginate hydrogel preparation capable of ATP-responsive slow release of immunoadjuvant obtained in Step (5) of Example 2, local X-ray irradiation of tumor (at a dose of 8 Gray), and intravenous injection of immune checkpoint inhibitor PD1 antibody (ALG-Aapt/CpG+RT). The final concentration of sodium alginate in each treatment group was 10 mg/mL, the volume of intratumoral injection was 25 μL, and the dose of CpG-ODN per mouse was 15 μg. On days 7, 9, 11, and 13, the mice were irradiated at a dose of 2 Gray per tumor. The immune checkpoint inhibitor PD1 antibody was administered intravenously at a dose of 10 μg per mouse on Days 8 and 11. The treatment process is schematically shown in FIG. 11A.

FIGS. 11(B), (C), and (D) show the volumes of the primary tumor (B) and the distal tumor (C) over time and the survival rate (D) of mice over time during the experiment, respectively. The experimental results (FIG. 11), the tumor growth curve and corresponding statistical data show that aPD1 alone or RT alone has limited efficacy in inhibiting tumor growth on both sides. Since the RT-induced antitumor immune response is enhanced by ALG-Aapt/CpG, RT+ALG-Aapt/CpG treatment can eliminate 4 of the 6 localized tumors on the right, and significantly delay the growth of distant tumors (on the left). However, most distant tumors in this group still show rapid growth later. Notably, aPD1 further improves the efficacy of RT+ALG-Aapt/CpG, all local tumors on the right side and 5 out of 6 distant tumors on the left side were eliminated.

### Example 9: Use of Smart Sodium Alginate Hydrogel Preparation Capable of ATP-Responsive Slow Release of Immunoadjuvant in Enhancing Radiotherapeutic Effect for Primary Breast Cancer Tumors

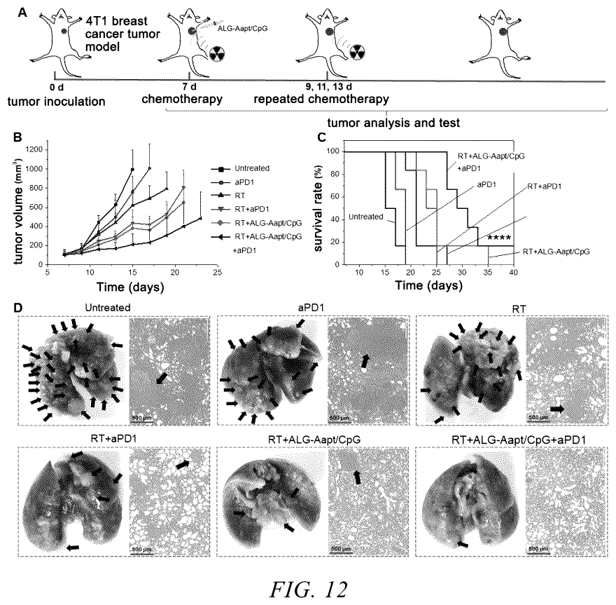

As shown, breast cancer tumors were inoculated subcutaneously on both sides of the abdomen of mice, and treatment was started on Day 7 after inoculation. Tumor-bearing BALB/c mice were randomly divided into the following 6 groups (6 mice in each group): Group 1: control group (Unteated); Group 2: intravenous injection of immune checkpoint inhibitor PD1 antibody (aPD1); Group 3: local X-ray irradiation of tumor (at a dose of 8 Gray) (RT); Group 4: intravenous injection of immune checkpoint inhibitor PD1 antibody (aPD1) and local X-ray irradiation of tumor (at a dose of 8 Gray) (RT); Group 5: intratumoral injection of smart sodium alginate hydrogel preparation (ALG-Aapt/CpG) capable of ATP-responsive slow release of immunoadjuvant obtained in Step (5) in Example 2 and local X-ray irradiation of tumor (at a dose of 8 Gray) (RT); Group 6: intratumoral injection of smart sodium alginate hydrogel preparation capable of ATP-responsive slow release of immunoadjuvant obtained in Step (5) of Example 2 and local X-ray irradiation of tumor (at a dose of 8 Gray), and intravenous injection of immune checkpoint inhibitor PD1 antibody (ALG-Aapt/CpG+RT). The final concentration of sodium alginate in each treatment group was 10 mg/mL, the volume of intratumoral injection was 25 μL, and the dose of CpG-ODN per mouse was 15 μg. On days 7, 9, 11 and 13, the mice were irradiated at a dose of 2 Gray per tumor. The immune checkpoint inhibitor PD1 antibody was administered intravenously at a dose of 10 μs per mouse on Days 8 and 11. The treatment process is schematically shown in FIG. 12(A).

The results are shown in FIG. 12. aPD1 alone or RT alone has limited efficacy in inhibiting tumor growth (FIG. 12B). As expected, treatment with RT+ALG-Aapt/CpG+aPD1 results in the most significant tumor growth inhibition and greatly prolongs the animal survival, and has better treatment response (FIGS. 12B-C), compared with RT+aPD1 or RT+ALG-Aapt/CpG. A mouse lung with a primary tumor volume exceeding 1000 mm³ was obtained on day 27, and the metastatic nodules were counted. Representative lung photographs show that despite dense metastatic nodules (indicated by black arrows) in the lungs of untreated mice, treatment with RT+ALG-Aapt/CpG+aPD1 can significantly inhibit the tumor metastasis (FIG. 12D). The pathological changes in representative lung tissues were further observed in hematoxylin-eosin (H&E) staining, confirming that the treatment with RT+ALG-Aapt/CpG+aPD1 can significantly inhibit the metastasis of 4T1 tumors to the lung.

While preferred embodiments of the present invention have been described above, the present invention is not limited thereto. It should be noted that several modifications and variations can be made by those of ordinary skill in the art, without departing from the technical principles of the present invention, which are also contemplated in the protection scope of the present invention.

1. Use of an immunoadjuvant-based hydrogel composition in preparing a sensitizing agent for surgical radiotherapy, wherein the immunoadjuvant-based hydrogel composition comprises sodium alginate and a water-soluble immunoadjuvant encapsulated in sodium alginate.

2. The use according to claim 1, wherein the immunoadjuvant-based hydrogel composition comprises sodium alginate, a tumor cell death marker aptamer, and a water-soluble immunoadjuvant with an extended sequence, wherein sodium alginate is covalently linked to the tumor cell death marker aptamer by a peptide bond, and the extended sequence is base-complementary to at least a part of a sequence of the tumor cell death marker aptamer.

3. The use according to claim 2, wherein the tumor cell death marker aptamer comprises an ATP aptamer, and a nucleotide sequence of the ATP aptamer comprises a sequence as shown in SEQ ID No: 1.

4. The use according to claim 2, wherein the extended sequence comprises a sequence as shown in SEQ ID No: 2.

5. The use according to claim 1, wherein the water-soluble immunoadjuvant is selected from the group consisting of a polynucleotide, CpG oligodeoxynucleotide, polyinosinic acid, polyICLC, lipopolysaccharide, muramyl peptide, lipoid A, a cytokine and any combination thereof.

6. The use according to claim 1, wherein the surgical radiotherapy sensitizing agent is used for the treatment of solid tumors.

7. An immunoadjuvant-based hydrogel composition, comprising sodium alginate, a tumor cell death marker aptamer, and a water-soluble immunoadjuvant with an extended sequence, wherein sodium alginate is covalently linked to the tumor cell death marker aptamer by a peptide bond, and the extended sequence is base-complementary to at least a part of the tumor cell death marker aptamer sequence.

8. The immunoadjuvant-based hydrogel composition according to claim 7, wherein the tumor cell death marker aptamer comprises an ATP aptamer.

9. The immunoadjuvant-based hydrogel composition according to claim 7, wherein the water-soluble immunoadjuvant is selected from the group consisting of a polynucleotide, CpG oligodeoxynucleotide, polyinosinic acid, polyICLC, lipopolysaccharide, muramyl peptide, lipoid A, a cytokine and any combination thereof.

10. A method for preparing an immunoadjuvant-based hydrogel composition according to claim 7, comprising steps of:
(1) reacting sodium alginate with an amino-modified tumor cell death marker aptamer in a solution, to obtain a sodium alginate-ATP aptamer conjugate; and
(2) subjecting the sodium alginate-ATP aptamer conjugate to a DNA hybridization reaction with the water-soluble immunoadjuvant with an extended sequence in a buffer, to obtain the immunoadjuvant-based hydrogel composition, wherein the extended sequence is base-complementary to at least a part of the tumor cell death marker aptamer sequence.
